(19) United States
(12) Patent Application Publication
(10) Pub. No.: US 2009/0280027 A1
(43) Pub. Date: Nov. 12, 2009

(54) Photocatalytic air treatment system and method

(76) Inventor: John J. Hayman, JR., (US)

Correspondence Address: BAKER, DONELSON, BEARMAN, CALDWELL & BERKOWITZ;Intellectual Property Department, Monarch Plaza, Suite 1600, 3414 Peachtree Rd., ATLANTA, GA, 30326, US

(21) Appl. No.: 12/384,237

(22) Filed: Apr. 2, 2009

Related U.S. Application Data
(63) Continuation-in-part of application No. 11/728,949, filed on Mar. 27, 2007.
(60) Provisional application No. 60/786,331, filed on Mar. 27, 2006.

(51) Int. Cl. A61L 9/20; B01J 19/12

(52) U.S. Cl. 422/4; 422/121

(57) ABSTRACT

A photocatalytic air treatment system, including an apparatus and method, is provided for killing and/or mineralizing bacteria, viruses, mold, fungi, spores, mycotoxins, allergens, and other similar microorganisms and agents and similar organic matter, and for oxidizing volatile organic compounds. The system comprises one or more reactor beds configured in one or more stages with each reactor bed including a plurality of photocatalyst-coated media substantially surrounding a plurality of sheathed ultraviolet light sources. Adjacent ultraviolet light sources are positioned to create killing zones of photocatalyst coated media that are irradiated with ultraviolet light from the ultraviolet light sources and in which an increased number of hydroxyl radicals are present.

### CROSS REFERENCE TO RELATED APPLICATIONS

This application is a continuation-in-part of, and claims the benefit of, U.S. patent application Ser. No. 11/728,949, filed Mar. 27, 2007; which in turn claims the benefit of U.S. Provisional Application Ser. No. 60/786,331, filed Mar. 27, 2006.

### FIELD OF THE INVENTION

The present invention relates to air treatment systems. In a more specific aspect, this invention relates to air treatment systems and methods using a photocatalytic reaction (a) to kill and/or mineralize bacteria, viruses, mold, fungi, spores, mycotoxins, allergens and other similar microorganisms and agents and similar organic matter and (b) to oxidize volatile organic compounds.

For purposes of this application, the term “microorganisms” will be used to collectively refer to such bacteria, viruses, mold, fungi, spores, mycotoxins, allergens and other similar microorganisms and agents.

### BACKGROUND OF THE INVENTION

For many years, people have suffered adverse physical effects (such as infections and allergic reactions) that have been knowingly or unknowingly either caused by or related to exposure to microorganisms. In attempts to reduce the suffering caused by such microorganisms, the medical community and pharmaceutical companies have directed substantial resources toward developing various drugs and other forms of medical treatments, but without overwhelming success. In other attempts to reduce the suffering, inventors and manufacturers have developed various devices that are targeted at killing such microorganisms.

Some devices, including those sometimes found in physician's offices, hospitals or other medical facilities, utilize ultraviolet light to treat air through which the ultraviolet light passes. Examples of devices which utilize an ultraviolet light source are bulbs, lamps and light emitting diodes. Unfortunately, such ultraviolet light devices have not been overly successful, at least in part due to their reliance on a sufficient amount of photons of ultraviolet light to heat the microorganisms as a mechanism to kill the microorganisms. (This reliance on photons of ultraviolet light for heating is considered in the industry as killing by contact time instead of killing by collision as in photocatalytic oxidation.) In many such ultraviolet light devices, treatment of air is attempted by allowing the air to move by natural or forced convection through the ultraviolet light spectra produced by ultraviolet light sources, such as bulbs, lamps, LEDs, etc. By virtue of being entrained in the air, the microorganisms also pass through and come in contact with the photons of ultraviolet light which heat and potentially kill the microorganisms. However, because the exposure time of the microorganisms to sufficiently intense ultraviolet light is often too short in duration due to movement of the air, many of the microorganisms escape and survive without being hit by a sufficient number of photons to heat and kill the microorganisms.

Other devices that have been developed produce ozone or other oxygen derivatives (such as O₁₂) that may, in turn, react with various volatile organic compounds found in carpets, rugs and other items to create formaldehyde. Also, the ozone may create hydroxyl radicals that attack organic compounds, such as organic compounds found in the breathing passages and tissues of humans, animals and other ozone-sensitive materials.

Filtering devices are unable to kill or destroy microorganisms and/or volatile organic compounds. However, these devices tend to collect or filter particles of limited sizes.

Therefore, there exists in the industry a need for a system, including apparatuses and methods, for treating air to kill and/or mineralize various microorganisms and to oxidize volatile organic compounds, and that addresses the above-described and other problems of current systems.

### SUMMARY OF THE INVENTION

The present invention provides a photocatalytic air treatment system, including apparatuses and methods, for killing and/or mineralizing microorganisms and for oxidizing volatile organic compounds.

The present invention also provides a photocatalytic air treatment system, including apparatuses and methods, that utilizes ultraviolet light in connection with a photocatalytic reaction that causes the killing and/or mineralization of microorganisms and the oxidation of volatile organic compounds.

The present invention involves photocatalysis, which is the excitation of a catalyst in the presence of ultraviolet light. This process is known as photocatalytic oxidation (PCO). In photocatalysis, light is absorbed by an adsorbed substrate. In photogenerated catalysis, the photocatalytic activity depends on the ability of the catalyst to create electron-hole pairs, which generate free radicals such as hydroxyl radicals (—OH) able to undergo secondary reactions. Titanium dioxide when exposed to UV light does create an electron-hole pair, which generates free radicals such as hydroxyl radicals (—OH).

Hydroxyl radicals can be formed by other chemical reactions. Ozone recently has been in the news as a problem in indoor and outdoor air pollution. Ozone creates the same free hydroxyl radicals as does photocatalytic oxidation with one great exception, Hydroxyl radicals created from ozone are gaseous and travel randomly within the environment. They chemically attack people, pets, animals, vegetables, fruits, flowers, rubber, anything organic, and the free radicals of PCOs are held to the surface of the TiO₂ molecule at the electron-pair hole and are not allowed to become a free floating gas. Therefore, hydroxyl radicals created from photocatalytic oxidation stay within the photocatalytic oxidation reactor bed and cannot come in contact with people, pets, animals, vegetables, fruits and flowers, but can be specifically directed to process and oxidize unwanted volatile organic compounds and kill and/or mineralize microorganisms.

The photocatalytic air treatment system of this invention does not rely on contact time as do prior systems which use only photons of ultraviolet light. Additionally, by using photons of ultraviolet light to activate the photocatalyst substance and by relying on collisions, the system of this invention is significantly more effective than prior systems.

In a preferred embodiment, the photocatalytic air treatment system of the present invention comprises one or more reactor beds configured in one or more stages. Each reactor bed includes a plurality of photocatalyst coated media formed from substrate media which are coated with a photocatalyst substance. The substrate media are formed, for example, from a glass or glass-like material that is non-reactive with both the photocatalyst substance and any reaction enhancing substance. The glass or glass-like material induces the photocatalyst substance to form on each substrate media as a nano-particle structure rather than as a mere closely packed layer, which enables and causes the photocatalyst substance to be struck by photons of ultraviolet light from a variety of directions.

Each reactor bed includes a plurality of ultraviolet light sources (located in the reactor bed) that are substantially surrounded by photocatalyst coated media, such that photons of ultraviolet light emitted by the ultraviolet light sources are directed outwardly at the photocatalyst coated media. The ultraviolet light sources are generally located in one or more arrangements at positions relative to one another that create one or more volumes of photocatalyst coated media in which ultraviolet light produced by the ultraviolet light sources is incident thereon and in which the irradiance of the ultraviolet light contributed to the volumes by multiple ultraviolet light sources and incident on the photocatalyst coated media is at a maximum.

Each reactor bed includes a plurality of sheaths cooperatively located with the plurality of ultraviolet light sources, such that at least one sheath is intermediate to an ultraviolet light source and surrounding photocatalyst coated media.

The photocatalytic air treatment system of this invention further comprises a directional air-handling unit that forces or induces air to flow through at least one reactor bed. The photocatalytic air treatment system may further comprise a heating unit that heats the air being treated to reduce the humidity of the system. In certain areas of high relative humidity, heating can speed up the photocatalytic oxidation reaction as the byproducts include water vapor.

In the photocatalytic air treatment system of this invention, use of photocatalyst coated media in which the photocatalyst substance forms a nano particle membrane structure on such media dramatically increases the number of reaction sites available for microorganisms and volatile organic compounds to undergo an oxidation reaction with a hydroxyl radical produced as part of the photocatalytic reaction. By dramatically increasing the number of available reaction sites and increasing the number of such oxidation reactions that occur, the system's ability to treat air, to kill and/or mineralize microorganisms and to oxidize volatile organic compounds is greatly improved over other systems.

The system of this invention makes a positive impact on the environment by killing or mineralizing microorganisms and by oxidizing volatile organic compounds in amounts which are significantly greater than achieved with prior systems.

In the photocatalytic air treatment system of this invention, the volumes of photocatalyst coated media created by the arrangement of the ultraviolet light sources relative to one another have an increased level of photocatalytic reactivity due to the irradiance of the ultraviolet light incident on the photocatalyst coated media being at a maximum. Because the volumes have an increased level of photocatalytic reactivity, the volumes also have an increased number of hydroxyl radicals available to react in an oxidation reaction with microorganisms and volatile organic compounds entrained in the untreated air, thereby improving the system's ability to kill, mineralize or oxidize such microorganisms and volatile organic compounds. Additionally, the reduction of the humidity of the untreated air with a heating unit also increases the level of photocatalytic reactivity and the production of hydroxyl radicals with a similar effect on the system's ability to kill, mineralize, or oxidize microorganisms and volatile organic compounds.

In the photocatalytic air treatment system of this invention, the inclusion of a sheath for each ultraviolet light source in a reactor bed enables the ultraviolet light sources to be replaced without disturbing the photocatalyst coated media present in the reactor bed. In addition, the system's ability to include one or more reactor beds in a particular configuration allows customization of the system to produce desired levels of air treatment.

Other advantages and benefits of the present invention will become apparent upon reading and understanding the present specification when taken in conjunction with the appended drawings.

### BRIEF DESCRIPTION OF THE DRAWINGS

FIG. 1 is a schematic, top plan view of a photocatalytic air treatment system for treating air in accordance with a first embodiment of the present invention.

FIG. 2 is a schematic, sectional view of the reactor bed of the photocatalytic air treatment system of FIG. 1 taken along lines 2-2.

FIG. 3 is a schematic, partial sectional view of the reactor bed of the photocatalytic air treatment system of FIG. 1 taken along lines 3-3.

FIG. 4 is a schematic, top plan view of a photocatalytic air treatment system for treating air in accordance with a second embodiment of the present invention.

FIG. 5 is a schematic, sectional view of the reactor bed of the photocatalytic air treatment system of FIG. 4 taken along lines 5-5.

FIG. 6 is a schematic, top plan view of a reactor bed of a photocatalytic air treatment system for treating air in accordance with a third embodiment of the present invention.

FIG. 7 is a schematic, sectional view of the reactor bed of the photocatalytic air treatment system of FIG. 6 taken along lines 7-7.

FIG. 8 is a schematic, top plan view of a photocatalytic air treatment system for treating air in accordance with a fourth embodiment of the present invention.

FIG. 9 is a schematic, top plan view of a photocatalytic air treatment system for treating air, in accordance with a fifth embodiment of the present invention, having multiple stages of air treatment.

### DETAILED DESCRIPTION OF THE INVENTION

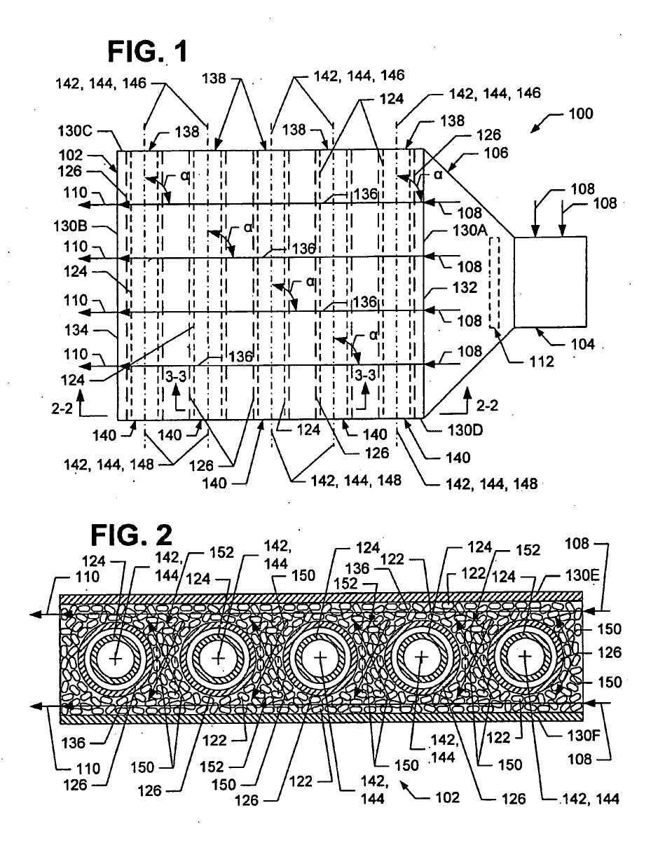

Referring now to the drawings in which like numerals represent like elements or steps throughout the several views, FIG. 1 is a schematic, top plan view of a photocatalytic air treatment system 100, according to a first embodiment of the present invention, for treating air by killing and/or mineralizing microorganisms and for oxidizing volatile organic compounds that may be present in the air. The photocatalytic air treatment system 100 comprises a reactor bed 102, an air-handling unit 104 and a transition member 106 interposed between and connected to the reactor bed 102 and the air-handling unit 104. The air-handling unit 104 is adapted to pull untreated air 108 (i.e., indicated by arrows 108) from the environment in which the photocatalytic air treatment system 100 is present or from another source and to direct the untreated air 108 into the reactor bed 102 via the transition member 106.

Generally, the air-handling unit 104 comprises a fan or blower that directs or blows untreated air 108 into the reactor bed 102 at a static pressure and volumetric flow rate selected and sufficient to overcome the static pressure drop caused by the reactor bed 102 during flow of the untreated air 108 through the reactor bed 102. This produces a desired volumetric rate of treated air 110 (i.e., indicated by arrows 110) exiting the system 100, and achieves a desired quality of air treatment as determined by measuring (e.g., in parts per thousand, million or billion) the quantity of killed, mineralized or oxidized microorganisms and volatile organic compounds present in the treated air 110 relative to the quantity of the microorganisms and volatile organic compounds present in the untreated air 108. The transition member 106 is configured to direct untreated air 108 exiting the air-handling unit 104 into the reactor bed 102 for treatment.

Generally, the transition member 106 comprises a plenum, duct or similar structure configured to direct and distribute the untreated air 108 into the reactor bed 102 in a manner that provides a substantially equal flow of untreated air 108 to all parts of the reactor bed 102. The air-handling unit 104 may optionally be connected directly to the reactor bed 102 without any transition member 106. The air-handling unit 104 may be positioned relative to the reactor bed 102 and operated in a manner that induces a flow of untreated air 108 through the reactor bed 102 instead of forcing a flow of untreated air 108 through the reactor bed 102.

In the air treatment system of the present invention, untreated air can be introduced into the reactor bed at a flow rate from about 0.01 to about 350 cubic feet per minute. A preferred rate of flow of the untreated air is from about 1 to about 100 cubic feet per minute, with the most preferred flow rate being from about 2 to about 40 cubic feet per minute.

The photocatalytic air treatment system 100 may further comprise, as illustrated in FIG. 1, a heating element 112 adapted to heat the untreated air 108 prior to entering into the reactor bed 102. Generally, the heating element 112 comprises an electric resistance heater, heating strip or other suitable device for raising the temperature of the untreated air 108 above its temperature when entering the air-handling unit 104. By raising the temperature of the untreated air 108 before entering the reactor bed 102, the reaction rate of the photocatalytic reaction (described in more detail below) is increased and the production of hydroxyl radicals is also increased, thereby enhancing the probability that microorganisms and/or volatile organic compounds will come into contact with a hydroxyl radical and undergo an oxidation reaction that kills, mineralizes or oxidizes the microorganisms and/or volatile organic compounds.

Further, by raising the temperature of the untreated air 108, the humidity level of the untreated air 108 is reduced, and the reaction rate of the photocatalytic reaction is increased with similar effects resulting as those due to the increase in temperature of the untreated air 108. However, while the operation of a heating element 112 is beneficial to the system's overall quality of air treatment, the photocatalytic air treatment system 100 need not include a heating element 112 to achieve substantial reductions in the quantities of volatile organic compounds and microorganisms before exiting the system 100.

The photocatalytic air treatment system of this invention is an enclosed system with the exception of the portion for entry of untreated air and the portion for discharge of treated air. Although not shown in certain Figures, the enclosed system includes top, bottom and side enclosures, which may be of metal or other suitable material.

As shown in FIG. 2, the reactor bed 102 comprises a plurality of photocatalyst is coated media 122 that are within the reactor bed 102. The photocatalyst coated media 122 are arranged in a substantially random manner such that air passing through the reactor bed 102 collides with, or comes into contact with, many photocatalyst coated media 122, thereby increasing the amount of time that microorganisms and volatile organic compounds present in the untreated air 108 are in contact with the photocatalyst coated media 122 and increasing the system's ability to kill, mineralize or oxidize the microorganisms and volatile organic compounds. The photocatalyst coated media 122 are also arranged to increase the number of photons of ultraviolet light that strike the media 122. Each photocatalyst coated media 122 includes a substrate media that is coated, at least in part, on the surface with a photocatalyst substance that undergoes a photocatalytic reaction when exposed to ultraviolet light. The substrate media can also be coated on all surfaces, including the hollow center and all internal and external sides. The photocatalytic reaction produces hydroxyl radicals on the surface of the substrate media that are available to combine, in an oxidation reaction, with microorganisms and volatile organic compounds present in the untreated air 108.

Generally, in the embodiments described in this application, the photocatalyst substance comprises titanium dioxide, but may also comprise other substances alone or in combination with titanium dioxide. Thus, the photocatalyst substance may further comprise a reaction enhancing substance (sometimes referred to as an “enhancer”) that increases the reaction rate of the photocatalytic reaction, thereby causing the production of an increased number hydroxyl radicals available for an oxidation reaction as described above. In the embodiments of this invention, the photocatalyst substance may further comprise an enhancer including zirconium dioxide in a quantity of about five to about twenty percent (about 5%-about 20%) of the total photocatalyst substance. However, the scope of the present invention includes the incorporation and use of enhancing substances other than zirconium dioxide.

The substrate media of the plurality of photocatalyst coated media 122 are selected to have shapes or forms providing a substantial amount of surface area for coating with a photocatalyst substance and for supplying sites for hydroxyl radicals to be exposed to untreated air 108 flowing through the reactor bed 102. By utilizing substrate media having shapes that provide maximal surface area, the amount of photocatalyst substance that is applied to and present on the substrate media is increased relative to the amount that may be applied to other shapes. Having shapes (including hollow cylinders) that provide maximal surface area also results in complementary increases occurring in the number of hydroxyl radicals that are created by the photocatalytic reaction of the photocatalyst substance with ultraviolet light and in the number of hydroxyl radicals that are actually contacted by and undergo an oxidizing reaction with microorganisms and volatile organic compounds present in the untreated air 108.

In accordance with the present invention, the substrate media are generally of a tubular shape or form somewhat similar to hollow cylindrical rings. The substrate media are coated at least partially with a photocatalyst substance on their inner and outer surfaces which provides a substantial amount of surface area for the creation, residence and reaction of hydroxyl radicals. In the present invention, the substrate media of the photocatalyst coated media 122 may have a cylindrical, spherical, toroidal, polyhedrical, round, hollow or other shape or form. Furthermore, in the present invention, the substrate media of the photocatalyst coated media 122 may have a plurality of different shapes or forms.

In the present invention, the substrate media of the plurality of photocatalyst coated media 122 are formed from a material (1) that does not react with (e.g., is inert relative to) the photocatalyst substance and (2) that induces the applied photocatalyst substance to form a nano particle membrane structure or coating on the top surface(s) of the substrate media instead of forming only a non-reactive, closely packed layer. By virtue of the photocatalyst substance forming a nano particle membrane structure or coating, the number of potential sites and surface area for photocatalysis to occur and for the concomitant creation of hydroxyl radicals for contact with and oxidation of microorganisms and volatile organic compounds is dramatically increased over the number of potential sites and surface area that would otherwise be available if the material of the substrate media did not induce the formation of a nano particle membrane structure.

Generally, the substrate media comprises bora silica glass, quartz glass or other materials (such as fiberglass, woven media or other media) that can be coated with a photocatalytic substance which can form a nano particle membrane structure are also considered to be within the scope of the present invention. The scope of the present invention includes other materials for substrate media that are normally reactive with the photocatalyst substance, but that are pre-coated with another substance that renders the materials non-reactive with the photocatalyst substance prior to being coated with the photocatalyst substance. Such reactive materials include, for example, materials commonly classified as plastics, metals and ceramics.

According to the present invention, the photocatalytic air treatment system 100 further comprises a plurality of ultraviolet light sources 124 that are positioned to emit photons of ultraviolet light at the photocatalyst coated media 122 and at the photocatalyst substance to cause a photocatalytic reaction of the photocatalyst substance to occur, hydroxyl radicals to be generated by the photocatalytic reaction and oxidation reactions of the hydroxyl radicals, microorganisms and volatile organic compounds to occur. The ultraviolet light sources 124 are generally adapted to produce light having one or more wavelengths within the ultraviolet portion of the electromagnetic spectrum. However, the scope of the present invention should be understood as including ultraviolet light sources 124 that may produce other light having one or more wavelengths that are not within the ultraviolet portion (e.g., wavelengths greater than 400 mn) of the electromagnetic spectrum.

In the air treatment system of the present invention, the ultraviolet light sources are adopted to produce light having a wavelength from about 180 to about 390 nanometers, preferably from about 240 to about 260 nanometers. A wavelength of about 254 nanometers is especially preferred. In this invention, the separate ultraviolet light sources 124 can produce light having different wavelengths.

Also, the ultraviolet light sources 124 are generally embodied in the form of elongated, tubular T5 lamps or bulbs that generate an irradiance of at least 3 microwatts per square centimeter, and preferably from about 3 to about 40 microwatts per square centimeter. The ultraviolet light sources are selected to provide the highest irradiance possible for the specific air being treated.

In other embodiments of the present invention, the ultraviolet light sources 124 may produce different levels of irradiance and may be embodied in other forms including, for example, ultraviolet lamps or bulbs having non-tubular or other shapes and ultraviolet light emitting diodes (LEDs). In still other embodiments of the present invention, the ultraviolet light sources 124 may be replaced by other devices, such as lamps or bulbs other than ultraviolet fluorescent lamps or bulbs; non-ultraviolet light emitting diodes; waveguides that increase surface areas and direct ultraviolet light and any energy light source that activates a photocatalyst; mercury vapor lamps (especially high pressure mercury vapor lamps); and microwave sources that are operable to provide a similar amount of energy as ultraviolet light sources 124 and/or that are operable to provide a sufficient amount of energy to the photocatalyst substance to cause photocatalysis and the photocatalytic reaction to occur. The scope of the present invention also includes reactor beds 102 having any number of lamps and/or bulbs, lamps and/or bulbs having the same or different sizes in terms of diameter and length, lamps and/or bulbs having the same or different wattages and/or any combination of the foregoing.

The lamps and/or bulbs useful in this invention can be any shape, size or wattage, with or without sheaths.

In the present invention, the reactor bed 102 further comprises a plurality of sheaths 126 for receiving the ultraviolet light sources 124 and for separating the ultraviolet light sources 124 from the photocatalyst coated media 122 that substantially surround the sheaths 126 and ultraviolet light sources 124. By separating the ultraviolet light sources 124 from the photocatalyst coated media 122 with intermediate sheaths 126, the ultraviolet light sources 124 may be inserted into and removed from the reactor bed 102 (i.e., inserted and removed from the sheaths 126) without coming into contact with the photocatalyst coated media 122, thereby making replacement of the ultraviolet light sources 124 much easier and less time consuming whenever such replacement is necessary. (When the lamps are being changed, the operator does not come into contact with the reactor bed which may have microorganisms that have not been killed or mineralized.)

The sheaths 126 are generally formed from a quartz or quartz-like material that enables ultraviolet light from the ultraviolet light sources 124 to pass through substantially unaffected and that does not react with the photocatalyst substance of the photocatalyst coated media 122. Although the embodiments of the present invention described in this application include a plurality of sheaths 126, the scope of the present invention includes other embodiments that do not include such sheaths 126. Even though the photocatalytic air treatment system 100 of the present invention is illustrated by different embodiments having particular numbers of ultraviolet light sources 124 and/or sheaths 126, the scope of the present invention comprises other embodiments having different numbers and arrangements of ultraviolet light sources 124 and/or sheaths 126.

The reactor bed 102 is formed from a plurality of panels 130 that confine the photocatalyst coated media 122 and that define a generally rectangular shape when viewed in top plan view as shown in FIG. 1. (However, the reactor bed 102 can be of shapes other than rectangular, such as square, round, oval, triangular, etc.) A first opposed pair of panels 130A, 130B respectively define an air inlet 132 and an air outlet 134 of the reactor bed 102. Panel 130A is generally formed from a perforated or mesh-like material suitable to confine the photocatalyst coated media 122″ and is adapted to receive untreated air 108 from transition member 106 and to allow the untreated air 108 to pass through and into the reactor bed 102. Panel 130B is similarly formed from a perforated or mesh-like material suitable to confine the photocatalyst coated media 122 and is adapted to receive treated air 110 from the reactor bed 102 and to allow the treated air 110 to pass through and to exit the photocatalytic air treatment system 100.

Second and third opposed pairs of such panels 130C, 130D, 130E, 130F are generally formed from a material that is not air permeable and are adapted to direct untreated air 108 from the air inlet 132 in a predominant or primary direction (e.g., designated by arrows 136) toward the air outlet 134. The reactor bed 102 may comprise a removable chamber or cartridge that may be disconnected and/or removed from fluid communication with the transition member 106 and air handling unit 104, so that the chamber or cartridge can be replaced after the elapse of an appropriate period of time (for example, one year) with a new or reconditioned removable chamber or cartridge having an identical or similar reactor bed 102 and enclosure 120, and having new or refurbished ultraviolet light sources 124, sheaths 126 and/or photocatalyst coated media 122.

Additionally, in this invention, one or more removable and replaceable filters (such as a HEPA filter—High Efficiency Particulate Air filter, % HEPA all-inclusive filter, carbon filter or carbon bed filter) may be connected in communication with the reactor bed 102 (whether comprising a removable chamber or not) generally at either the intake or exhaust so that the air also passes through such filter(s) to receive further treatment and conditioning. In still other embodiments, one or more filters as described above may be integral with the reactor bed 102 to define and form a removable and replaceable chamber or cartridge of the photocatalytic air treatment system 100. In other embodiments, one or more filters as described above may be positioned at various other locations within the air flow path of the photocatalytic air treatment system 100.

A HEPA filter is a specially constructed filter membrane that allows a high volume of air flow and stops small particles of a pre-determined size from passing through, preferably with a trapping efficiency greater than 99.99 percent for particles of 0.3 micrometers in diameter. A carbon filter or carbon bed filter is a specially constructed filter media, either of a carbon mesh-type material or of carbon particulate matter (e.g., granules, pellets, etc.) that has the ability to absorb odors. A carbon filter or carbon bed filter may be a useful addition to the system of this invention to absorb odors, such as non-organic odors.

The sheaths 126 extend substantially between the second opposed pair of panels 130C, 130D at locations corresponding respectively to holes 138, 140 defined by panels 130C, 130D. Each sheath 126 has a generally elongate sleeve-like shape with a longitudinal centerline 142 and is sized to receive a corresponding ultraviolet light source 124 having a longitudinal centerline 144 such that longitudinal centerlines 142, 144 are substantially collinear. The holes 138, 140 in respective panels 130C, 130D have longitudinal centerlines 146, 148 that are also substantially collinear with the longitudinal centerlines 142, 144 of the respective sheaths 126 and ultraviolet light sources 124. By virtue of such arrangement and alignment of respective sheaths 126, holes 138, 140 and ultraviolet light sources 124, the ultraviolet light sources 124 may be easily inserted into and removed from the sheaths 126 as necessary for assembly, disassembly and/or maintenance.

A quartz (or quartz-like) sheath 126 facilitates and allows the replacement of the lamp or bulb without a collapse of the catalyst bed.

A precision grommet may be used to set the distance between the ultraviolet light sources.

In accordance with the present invention, the sheaths 126 and ultraviolet light sources 124 are arranged in a single row with the centerlines 142, 144 of the sheaths 126 and ultraviolet light sources 124 defining angles (alpha) with the primary direction 136 of air travel through the reactor bed 102. All of the angles generally (but not necessarily) have substantially the same angular measure which is typically between 0 degrees and 180 degrees. According to the embodiment shown in FIGS. 1 and 2, the angles have an angular measure of approximately 90 degrees, such that the primary direction 136 of air travel through the reactor bed 102 is substantially perpendicular to the longitudinal centerlines 142, 144 of the sheaths 126 and ultraviolet light sources 124. By virtue of the primary direction 136 of air flow through the reactor bed 102 being substantially transverse to (and, in the first embodiment, substantially perpendicular to) the longitudinal centerlines 142, 144 of the sheaths 126 and ultraviolet light sources 124, the sheaths 126 and ultraviolet light sources 124 act as obstructions (or baffles) to the flow of air through the reactor bed 102, create air turbulence within the reactor bed 102 and cause portions of the air attempting to flow through the reactor bed 102 in the primary direction 136 to be diverted into secondary directions (e.g., indicated by arrows 150); see FIG. 2.

By diverting portions of the air traveling through the reactor bed 102 into secondary directions 150, the residence time of such air portions in the reactor bed 102 is dramatically increased and, consequentially, there is a substantial increase in the number of microorganisms and volatile organic compounds present in such air portions that come into contact with and are killed, mineralized or oxidized by hydroxyl radicals produced by the photocatalytic reaction. The air flow through the reactor bed 102 can be reversed 180 degrees.

The direction of air travel in the system of this invention can be altered (i.e., multi-directional) depending on the arrangement of the ultraviolet light sources and other components. For example, by changing the cavitation of the fan, the direction of air flow 136 and 150 can be reversed.

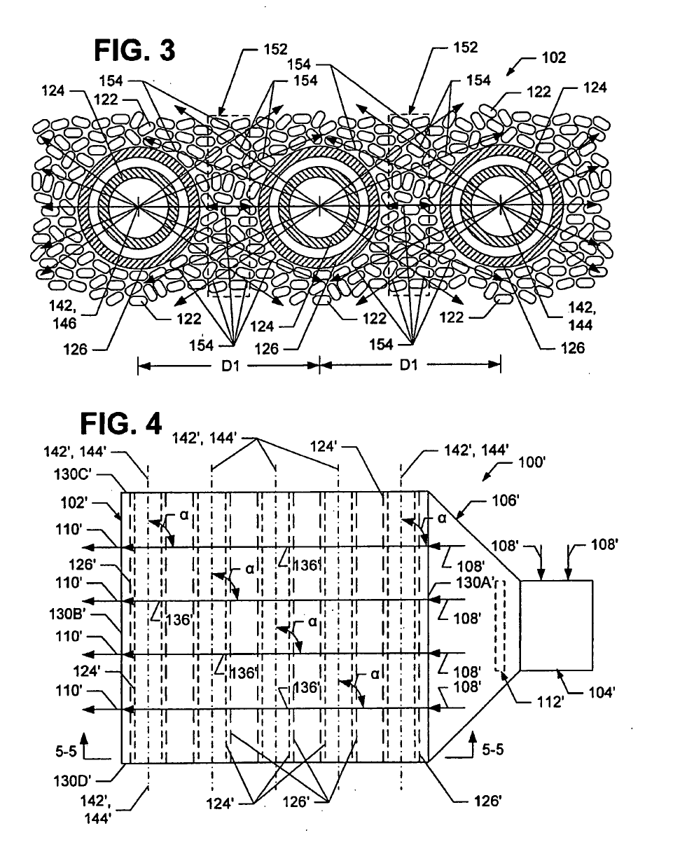

As illustrated in FIG. 3, the sheaths 126 of the reactor bed 102 are generally positioned adjacent to one another such that the corresponding ultraviolet light sources 124 are also generally positioned adjacent to one another and define a center-to-center distance, D1 from about 0.92 inches to about 3.17 inches. The distance between sheaths 126 is about 0.17 inches to about 3.00 inches. In such an arrangement, portions of the photocatalyst coated media 122 reside between adjacent sheaths 126 and, hence, between adjacent ultraviolet light sources 124. Further, many of such photocatalyst coated media 122 reside within an elongate volume 152 which extends between adjacent ultraviolet light sources 124 and panels 130C, 130D in which the irradiance of the ultraviolet light 154 striking the members 122 corresponds to the combined irradiance of the ultraviolet light 154 emitted outwardly by the adjacent ultraviolet light sources 124. Because the irradiance of the ultraviolet light 154 striking the photocatalyst coated media 122 within such elongate volumes 152 is higher than the irradiance of the ultraviolet light 154 striking photocatalyst coated media 122 not within such elongate volumes 152, the number of hydroxyl radicals created by the photocatalytic reaction is greater within such elongate volumes 152 and, hence, the number of microorganisms and volatile organic compounds present in air passing through such elongate volumes 152 that come into contact with and undergo an oxidation reaction with hydroxyl radicals is substantially greater. As a consequence, each such elongate volume 152 is sometimes referred to as a “killing zone”. At the center of each such elongate volume 152 is a concentrated “killing zone”.

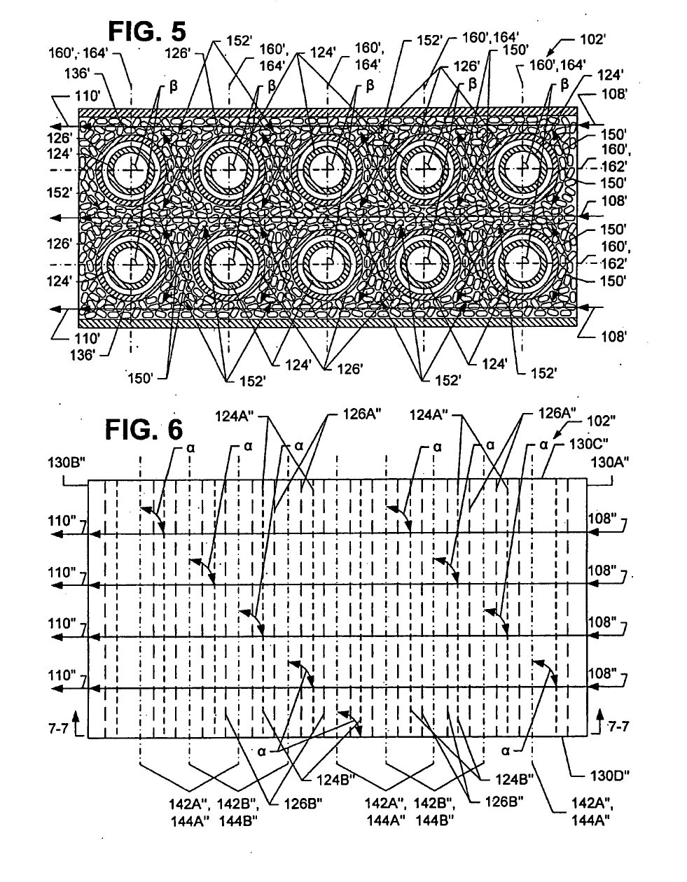

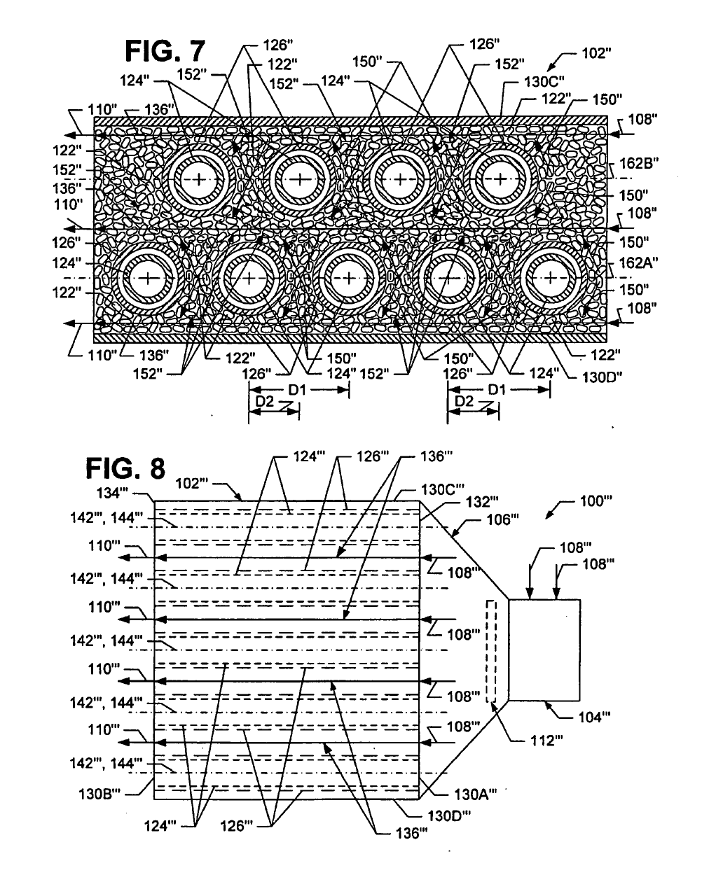

Broadly defined, the killing zone is generally the center area where photon energy (in microwatts per centimeter square) from 4 UV bulbs converges, which results in a cross-fire of photons. The killing zone is created by the photon energy generating numerous hydroxyl radicals which are confined within the killing zone. By numerous hydroxyl radicals is meant millions, billions, sometimes even trillions, of hydroxyl radicals. The photon energy within the killing zone is 104 power as compared to any other side of a single UV bulb. The killing zone can be seen in FIGS. 5 and 7.

As stated above in this application, each ultraviolet light source 124 is operable to produce ultraviolet light 154 having an irradiance of at least 3, and preferably from about 3 to about 40, microwatts per square centimeter. The number of hydroxyl radicals created by the photocatalytic reaction is proportional to the irradiance of the ultraviolet light 154 striking the photocatalyst coated media 122 within such elongate volumes 152.

FIG. 4 displays a schematic, top plan view of a photocatalytic air treatment system 100′ in accordance with a further embodiment of the present invention that is substantially similar to the embodiment shown in FIG. 1. However, in this embodiment, the plurality of ultraviolet light sources 124′ and corresponding plurality of sheaths 126′ are arranged within the reactor bed 102′ in a row and column matrix 160′ having multiple rows 162′ and multiple columns 164′. The row and column matrix 160′ is more clearly illustrated in FIG. 5. The rows 162′ of ultraviolet light sources 124′ and corresponding sheaths 126′ define angles (beta) with the columns 164′ of ultraviolet light sources 124′ and corresponding sheaths 126′. Generally, all of the angles have the same angular measure. Also generally, each angle has an angular measure of 90 degrees. In other embodiments of the present invention, the angles may have the same or different angular measures and/or angular measures other than 90 degrees.

In a manner similar to that of the reactor bed 102 of the embodiment shown in FIG. 1, the primary direction 136′ of air travel through the reactor bed 102′ is transverse to the longitudinal centerlines 142′, 144′ of the respective sheaths 126′ and ultraviolet light sources 124′. However, the row and column matrix 160′ of ultraviolet light sources 124′ and sheaths 126′ of the second embodiment advantageously produces an increased number of obstructions (or baffles) to the flow of air through the reactor bed 102′ than are present in the reactor bed 102 of the photocatalytic air treatment system 100 of the embodiment shown in FIG. 1. The row and column matrix 160′ also advantageously results in the reactor bed 102′ having an increased number of adjacent ultraviolet light sources 124′ and an increased number of elongate volumes 152′ extending between such adjacent ultraviolet light sources 124′ and panels 130C′, 130D′ in which the irradiance of the ultraviolet light striking the photocatalyst coated media 122′ corresponds to the combined irradiance of the ultraviolet light emitted outwardly by such adjacent ultraviolet light sources 124′. Due at least in part to the increased number of such elongate volumes 152″, the arrangement of the ultraviolet light sources 124′ of the second embodiment increases the number of microorganisms and volatile organic compounds present in untreated air 108′ that come into contact with and undergo an oxidation reaction with hydroxyl radicals, thereby increasing the number of microorganisms and volatile organic compounds that are killed, mineralized and/or oxidized.

FIG. 6 displays a schematic, top plan view of a photocatalytic air treatment system 100″ in accordance with a further embodiment of the present invention that is substantially similar to the embodiment shown in FIG. 4. The plurality of ultraviolet light sources 124″ and corresponding plurality of sheaths 126″ are arranged within the reactor bed 102″ in multiple rows 162″ and multiple columns 164″, and the primary direction 136″ of air flow through the reactor bed 102″ is transverse to the longitudinal centerlines 142″, 144″ of the respective sheaths 126″ and ultraviolet light sources 124″. The longitudinal centerlines 142″, 144″ of the sheaths 126″ and ultraviolet light sources 124″ define angles (alpha) with the primary direction 136 of air flow through the reactor bed 102″. Generally, the angles have a measure of approximately 90 degrees.

However, in a further embodiment shown in FIG. 7, the ultraviolet light sources 124″ and sheaths 126″ of the second row 162B″ are offset relative to the ultraviolet light sources 124″ and sheaths 126″ of the first row 162A″ by an offset distance D2. Generally, the offset distance D2 has a measure of approximately one-half of the center-to-center distance D1 between the ultraviolet light sources 124″ and sheaths 126″ of the first row 162A″. By offsetting the ultraviolet light sources 124″ and sheaths 126″ of the second row 162B″, the level of turbulence in the air flowing through the reactor bed 102″ is increased with more portions of the air traveling in secondary directions 150″. The nearer adjacency of the ultraviolet light sources 124″ of the second row 162B″ to multiple ultraviolet light sources 124″ of the first row 162A″ produces an increased number of elongate volumes 152″ between such ultraviolet light sources 124″ and within the reactor bed 102″ in which the irradiance of the ultraviolet light striking the photocatalyst coated media 122″ corresponds to the combined irradiance of the ultraviolet light emitted outwardly by such adjacent ultraviolet light sources 124″.

FIG. 8 shows a photocatalytic air treatment system 100′″ for treating air according to a further embodiment of the present invention. As shown in FIG. 8, the photocatalytic air treatment system 100′″ comprises components substantially similar to those of the embodiment shown in FIG. 1. The photocatalytic air treatment system 100′″ comprises a reactor bed 102′″, an air-handling unit 104′″, and a transition member 106′″ coupled between the reactor bed 102′″ and air-handling unit 104′″ for guiding the flow of untreated air 108′″ from the air-handling unit 104′″ into the reactor bed 102′″. Similar to the reactor bed 102 of the embodiment shown in FIG. 1, the reactor bed 102′″ of this embodiment includes a plurality of sheaths 126′″ for receiving a corresponding plurality of ultraviolet light sources 124′″ and for separating the ultraviolet light sources 124′″ from the photocatalyst coated media 122′″ that substantially surround the sheaths 126′″ and ultraviolet light sources 124′″. However, in this embodiment, the longitudinal centerlines 142″, 144″ of the sheaths 126′″ and ultraviolet light sources 124′″ are substantially parallel to the primary direction 136′″ of air flow through the reactor bed 102′″. As a consequence, a predominant portion of the untreated air 108′″ entering the reactor bed 102′″ travels through at least one elongate volume 152′″ located between adjacent ultraviolet light sources 124′″ and, therefore, a substantial number of microorganisms and volatile organic compounds present in untreated air 108″ come into contact with hydroxyl radicals and are killed, mineralized and/or oxidized.

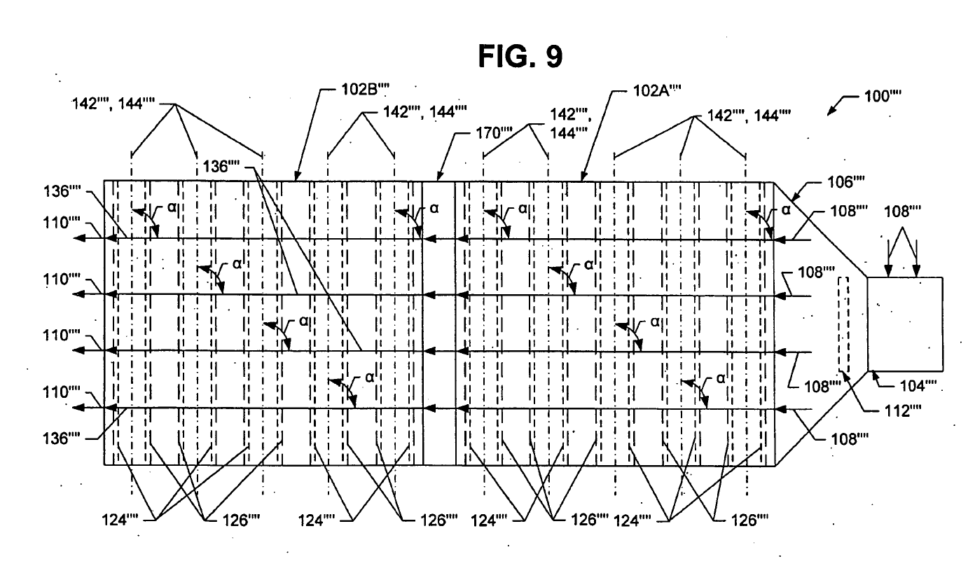

FIG. 9 shows a photocatalytic air treatment system 100″″ in accordance with a further embodiment of the present invention. The photocatalytic air treatment system 100″″ includes multiple stages of air treatment instead of a single stage of air treatment as in the photocatalytic air treatment system 100 of the embodiment shown in FIG. 1. Due at least in part to the use of multiple stages of air treatment, the photocatalytic air treatment system 100″″ of this embodiment is capable of killing, mineralizing and/or oxidizing larger numbers of microorganisms and volatile organic compounds than the photocatalytic air treatment system 100 of the embodiment shown in FIG. 1.

In addition to similar components of the photocatalytic air treatment system 100 of the embodiment shown in FIG. 1, the photocatalytic air treatment system 100″″ of the embodiment shown in FIG. 9 comprises a first reactor bed 102A″″ that is adapted to perform a first stage of air treatment and a second reactor bed 102B″″ that is adapted to perform a second stage of air treatment. The first reactor bed 102A″″ and second reactor bed 102B″″ are connected by a coupling member 170″″ for directing treated air 110″″ from the first reactor bed 102A″″ into the second reactor bed 102B″″ for further treatment. Generally, coupling member 170″″ includes a plenum, duct or other similar apparatus. Alternatively, the first reactor bed 102A″″ can be abutted and directly connected to the second reactor bed 102B″″ without coupling member 170″″.

In the photocatalytic air treatment system 100″″ shown in FIG. 9, the first and second reactor beds 102A″″, 102B″″ each comprise a plurality of ultraviolet light sources 124″″ and a corresponding plurality of sheaths 126″″ having respective longitudinal centerlines 144″″, 142″″ that define angles relative to the primary direction 136″″ of air flow through the reactor beds 102″″. Generally, each angle has the same angular measure. Also generally, each angle has an angular measure within a range of 45 degrees to 135 degrees. Thus, the ultraviolet light sources 124″″ and sheaths 126″″ are oriented such that the primary direction 136″″ of air flow through each reactor bed 102″″ is substantially transverse to the longitudinal centerlines 144″″, 142″″ of the ultraviolet light sources 124″″ and sheaths 126″″. This creates air turbulence within the reactor beds 102″″, causes air to flow in secondary directions 150 (refer to FIG. 2) within the reactor beds 102″″, increases the residence time for microorganisms and volatile organic compounds within the reactor beds 102″″ and increases the number of microorganisms and volatile organic compounds that are killed, mineralized and/or oxidized.

Although the angular measure of angles is generally the same in both reactor beds 102″″ of the photocatalytic air treatment system 100″″, the scope of the present invention includes other embodiments in which the angular measure of angles is not the same. Therefore, the scope of the present invention includes photocatalytic air treatment systems 100″″ in which the primary direction 136″″ of the flow of air through one reactor bed 102″″ is substantially transverse to the longitudinal centerlines 144″″, 142″″ of the ultraviolet light sources 124″″ and sheaths 126″″, while the primary direction 136″″ of the flow of air through a second reactor bed 102″″ is substantially parallel to the longitudinal centerlines 144″″, 142″″ of the ultraviolet light sources 124″″ and sheaths 126″″.

Further, the scope of the present invention includes photocatalytic air treatment systems 100″″ in which the primary direction 136″″ of the flow of air through both reactor beds 102″″ is substantially parallel to the longitudinal centerlines 144″″, 142″″ of the ultraviolet light sources 124″″ and sheaths 126″″.

Before proceeding with a description of a method of operation of the photocatalytic air treatment system of the present invention, in other embodiments of the present invention at least one pair of the ultraviolet light sources have a distance between that is different from the distance between the ultraviolet light sources of other pairs of ultraviolet light sources. Additionally, in other embodiments of the present invention, the plurality of ultraviolet light sources are arranged in different arrangements such that the time required for moving air to travel or pass by all of the ultraviolet light sources is increased relative to the time required for moving air to travel or pass by all of the ultraviolet light sources of an arrangement in which the plurality of ultraviolet light sources are positioned substantially in a single row or single column. By virtue of arranging the plurality of ultraviolet light sources in an arrangement that causes such an increase in the time required for air to travel or pass by all of the ultraviolet light sources, the number of collisions (or contacts) between microorganisms and/or volatile organic compounds present in the air and the plurality of media of a reactor bed are also increased, thereby resulting in an increase in the number of microorganisms and/or volatile organic compounds that are killed, mineralized and/or oxidized.

In operation, the photocatalytic air treatment systems 100, 100′, 100″, 100′″, 100″″ of the various embodiments function according to substantially the same method. Therefore, although directed primarily to the photocatalytic air treatment system 100 of the embodiment shown in FIG. 1, the following description is generally also applicable to the other embodiments.

The photocatalytic air treatment system 100 is generally positioned within the environment in which air is to be treated and is connected to an appropriate electrical power supply. Once activated, the air-handling unit 104 pulls in untreated air 108 from the environment through an air intake and blows the untreated air 108 out through an exhaust and into the transition member 106 at an appropriate static pressure, velocity and volumetric flow rate. (Alternatively, the air-handling unit 104 can be repositioned or rearranged to pull the untreated air 108 in a reverse direction.) While traveling though the transition member 106, the untreated air 108 may be heated by heating element 112 to raise the temperature and reduce the relative humidity of the untreated air 108. By increasing the temperature and reducing the relative humidity of the air, the reaction rates of the photocatalytic and oxidation reactions occurring within the reactor bed 102 are increased, thereby resulting in an increased production of hydroxyl radicals and an increased number of microorganisms and volatile organic compounds being killed, mineralized and/or oxidized.

The transition member 106 then directs the untreated air 108 through a filter (such as a HEPA filter) if one is present and into the reactor bed 102 through air inlet 132. Once inside the reactor bed 102, the untreated air 108 flows through the photocatalyst coated media 122 and toward air outlet 134 in the primary direction 136 of air flow. (This system can also function properly by reversing the airflow 180 degrees.) However, as the untreated air 108 comes into contact with the sheaths 126, portions of the untreated air 108 are deflected and redirected in secondary directions 150 and into the elongate volumes 152 of photocatalyst coated media 122 located between adjacent ultraviolet light sources 124. In the elongate volumes 152, the untreated air 108 comes into contact with photocatalyst coated media 122 that has been exposed to ultraviolet light having an irradiance corresponding to the combined irradiance of the ultraviolet light 154 emitted outwardly by the adjacent ultraviolet light sources 124. Due at least in part to the photocatalyst coated media 122 of the elongate volumes 152 being exposed to a greater irradiance of ultraviolet light 154, the photocatalyst coated media 122 host a greater number of hydroxyl radicals to which microorganisms and volatile organic compounds come into contact. Upon such contact, the hydroxyl radicals react in an oxidation reaction with the microorganisms and volatile organic compounds causing them to be killed, oxidized and/or mineralized and improving the quality of the treated air 110.

The portions of the untreated air 108 that do not flow through an elongate volume 152 are also subjected to hydroxyl radicals present on photocatalyst coated media 122 with similar results, but they are not subjected to the same highly concentrated numbers of hydroxyl radicals that are present within the elongate volumes 152. The longer untreated air 108 is resident within the reactor bed 102, there is an increased statistical probability that the microorganisms and volatile organic compounds will come into contact with hydroxyl radicals and be killed, oxidized and/or mineralized, thereby improving the quality of the treated air 110.

After flowing through the photocatalyst coated media 122, treated air 110 exits the reactor bed 102 through outlet 134 and back into the environment of the photocatalytic air treatment system 100. Of course, if the photocatalytic air treatment system 100 has multiple stages of air treatment, the untreated air 108 flows through multiple reactor beds 102 before being reintroduced into the environment, thereby resulting in increased air treatment.

The photocatalytic air treatment system of the present invention may be utilized to treat air in a variety of applications. For example, the system can be used to treat air in environments which are controlled, not controlled or modified, such as by temperature, humidity, refrigeration, HVAC, electric and ambient environments. These environments may be found in commercial, industrial, residential, military, healthcare, lodging, hospitality, penal institutions, daycare facilities, educational institutions, food storage, public places, government locations and many other locations and environments.

In addition, the photocatalytic air treatment system may be used in connection with residential and commercial grade refrigerators, refrigeration systems and wine coolers to treat the air present within, supplied to or exhausted from those environments. In other implementations, the photocatalytic air treatment system may be utilized in conjunction with central and/or standalone air conditioning systems for residential and/or commercial use that may or may not include additional air handling units in order to treat the conditioned air supplied to residential and commercial rooms, buildings, facilities, and/or structures. The photocatalytic air treatment system of this invention may be used in connection with bathroom and/or vehicle air circulation, air exhaust and/or air conditioning systems to treat air provided to and/or exhausted from bathrooms and/or the passenger compartments of refrigerated tractor trailers, ships and planes.

The photocatalytic air treatment system of this invention may also be used with an air sanitizing module for an anesthesia machine to remove trace volatile organic compounds generated by anesthetic agents. The photocatalytic air treatment system may be designed and integrated into an existing portable air purifying device used in hospitals. The photocatalytic air treatment system of this invention may be incorporated into a ceiling or house fan for use in various rooms or structures, including rooms for infants. The photocatalytic air treatment system of this invention may be utilized with a hospital bed to provide onboard air treatment and/or purification. The photocatalytic air treatment system of this invention may also be utilized in connection with cages or other enclosures used to house and/or transport animals to treat air supplied and/or exhausted from those enclosures.

The system of this invention may be used in an open or closed environment.

The present invention has been described in detail with particular reference to certain embodiments, but variations and modifications can be made without departing from the spirit and scope of the present invention as defined in the following claims.

What is claimed is:

1. An apparatus for treating air using a photocatalytic reaction, wherein the apparatus comprises:
A. at least one reactor bed comprising (1) a plurality of media coated at least partially with a photocatalyst substance and (2) a plurality of ultraviolet light sources located within and substantially surrounded by the plurality of media,
wherein the plurality of media is located around and substantially between the ultraviolet light sources to receive ultraviolet light from the ultraviolet light sources which provide light having a wavelength from about 180 to about 390 nanometers, and
B. an air-handling unit in communication with the reactor bed and adapted to (1) move untreated air into and through the reactor bed and into contact with at least a portion of the plurality of photocatalyst-coated media and (2) subsequently move treated air from the reactor bed.

2. An apparatus as defined in claim 1, wherein a transition member is located between the air-handling unit and the reactor bed.

3. An apparatus as defined in claim 1, wherein each ultraviolet light source is positioned within a sheath which is located between the ultraviolet light source and the photocatalyst-coated media in the reactor bed, wherein increased collisions are created between the untreated air and the photocatalyst-coated media.

4. An apparatus as defined in claim 1, wherein at least one ultraviolet light source has a longitudinal axis extending in a first direction and is oriented such that air from the air-handling unit moves in a direction substantially transverse to the longitudinal axis.

5. An apparatus as defined in claim 1, wherein at least one ultraviolet light source has a longitudinal axis extending in a first direction and is oriented such that air from the air-handling unit moves in a direction substantially parallel to the longitudinal axis.

6. An apparatus as defined in claim 1, wherein the reactor bed has a generally rectangular shape.

7. An apparatus as defined in claim 1, wherein the ultraviolet light sources are positioned so the center-to-center distance from an ultraviolet light source to an adjacent ultraviolet light source is about 0.92 inches to about 3.17 inches.

8. An apparatus as defined by claim 3, wherein the sheaths are positioned so that the distance from a sheath to the adjacent sheath is about 0.17 inches to about 3.00 inches.

9. An apparatus as defined by claim 1, wherein microorganisms and volatile organic compounds contained in the air being treated are substantially killed, mineralized or oxidized.

10. An apparatus as defined in claim 1, wherein the reactor bed is enclosed by top, side and bottom panels.

11. An apparatus as defined by claim 10, wherein the killing and mineralizing of microorganisms and oxidizing of volatile organic compounds are achieved in a killing zone extending between adjacent ultraviolet light sources and the side panels of the reactor bed.

12. An apparatus as defined by claim 1, wherein killing and mineralizing of microorganisms and oxidizing of volatile organic compounds are concentrated in a killing zone located substantially equidistant from adjacent ultraviolet light sources.

13. An apparatus as defined by claim 1, wherein killing and mineralizing of microorganisms and oxidizing of volatile organic compounds are achieved in a killing zone created by hydroxyl radicals generated by the photocatalyst-coated media upon contact with photons from the ultraviolet light sources.

14. An apparatus as defined by claim 1, wherein the photocatalyst-coated media are cylindrical, spherical, toroidal, polyhedrical, round or hollow and are arranged to form a random path for the untreated air, wherein increased collisions are created between the untreated air and the photocatalyst-coated media.

15. A process for treating air using a photocatalytic reaction, wherein the process comprises moving untreated air into, through and in contact with at least one reactor bed comprising (1) a plurality of media coated at least partially with a photocatalyst substance and (2) a plurality of ultraviolet light sources located within and substantially surrounded by the plurality of media,
wherein the plurality of media receives ultraviolet light from the ultraviolet light sources to create a photocatalytic reaction by which microorganisms and volatile organic compounds in the air are substantially killed, mineralized or oxidized.

16. A process as defined by claim 15, wherein each ultraviolet light source is positioned within a sheath which is located between the ultraviolet light source and the photocatalyst-coated media in the reactor bed.

17. A process as defined by claim 15, wherein at least one ultraviolet light source has a longitudinal axis extending in a first direction and is oriented such that air from the air-handling unit moves in a direction substantially transverse to the longitudinal axis.

18. A process as defined by claim 15, wherein at least one ultraviolet light source has a longitudinal axis extending in a first direction and is oriented such that air from the air-handling unit moves in a direction substantially parallel to the longitudinal axis.

19. A process as defined by claim 15, wherein the ultraviolet light sources are positioned so the center-to-center distance from an ultraviolet light source to an adjacent ultraviolet light source is about 0.92 inches to about 3.17 inches.

20. A process as defined by claim 16, wherein the sheaths are positioned so that the distance from a sheath to the adjacent sheath is about 0.17 inches to about 3.00 inches.

21. A process as defined by claim 15, wherein the reactor bed is enclosed by top, side and bottom panels.

22. A process as defined by claim 21, wherein killing and mineralizing of microorganisms and oxidizing of volatile organic compounds are achieved in a killing zone extending between adjacent ultraviolet light sources and the side panels of the reactor bed.

23. A process as defined by claim 15, wherein killing and mineralizing of microorganisms and oxidizing of volatile organic compounds are concentrated in a killing zone located substantially equidistant from adjacent ultraviolet light sources.

24. A process as defined by claim 15, wherein each ultraviolet light source is positioned within a sheath which is located between the ultraviolet light source and the photocatalyst-coated media in the reactor bed, wherein the ultraviolet light source is replaceable without contacting the photocatalyst-coated media.

25. A process as defined by claim 15, wherein the photocatalyst-coated media are cylindrical, spherical, toroidal, polyhedrical, round or hollow and are arranged to form a random path for the untreated air, wherein increased collisions are created between the untreated air and the photocatalyst-coated media.

26. A process as defined by claim 15, wherein killing and mineralizing of microorganisms and oxidizing of volatile organic compounds are achieved in a killing zone created by hydroxyl radicals generated by the photocatalyst-coated media upon contact with photons from the ultraviolet light sources.
